(19) United States
(12) Patent Application Publication
(10) Pub. No.: US 2022/0127578 A1
(43) Pub. Date: Apr. 28, 2022

(54) NOVEL EXOSOME PRODUCTION METHOD AND APPLICATION THEREOF

(71) Applicant: EXOCOBIO INC., Seoul (KR)

(72) Inventors: Byong Seung CHO, Gunpo-si (KR); Dae Hyun HA, Seoul (KR)

(73) Assignee: EXOCOBIO INC., Seoul (KR)

(21) Appl. No.: 17/570,611

(22) Filed: Jan. 7, 2022

Related U.S. Application Data
(63) Continuation of application No. PCT/KR2020/007768, filed on Jun. 16, 2020.

(30) Foreign Application Priority Data
Jul. 30, 2019 (KR) 10-2019-0092117
Jun. 8, 2020 (KR) 10-2020-0068797

(51) Int. Cl. C12N 5/0775; A61K 8/98; A61Q 19/08

(52) U.S. Cl. CPC C12N 5/0667; A61K 8/981; C12N 2500/84; C12N 2501/25; C12N 2501/24; A61Q 19/08

(57) ABSTRACT

A method for producing exosomes is provided. The method includes culturing animal cells in a medium containing TNF-α and interferon-γ. This method is possible not only to increase production of exosomes to be isolated from a cell-derived conditioned medium, but also to exhibit an excellent effect of enhancing bioactivity of exosomes, such as increasing elastin synthesis of exosomes.

### CROSS REFERENCE

This application is a Bypass Continuation of International Application No. PCT/KR2020/007768 filed Jun. 16, 2020, claiming priority based on Korean Patent Application No. 10-2019-0092117 filed Jul. 30, 2019 and Korean Patent Application No. 10-2020-0068797 filed Jun. 8, 2020, the entire contents of which are incorporated herein by reference.

### TECHNICAL FIELD

The present invention relates to a method for producing exosomes, and specifically, to a method for producing exosomes, which can increase exosome production and/or enhance bioactivity of exosomes.

Moreover, the present invention relates to a method for increasing exosome production, a method for enhancing bioactivity of exosomes, and a medium therefor.

Additionally, the present invention relates to applications of exosomes having enhanced bioactivity, obtained by the production method as described above.

### BACKGROUND ART

Recently, there have been reports that cell secretomes contain various bioactive molecules that regulate cellular behaviors. In particular, cell secretomes contain ‘exosome’ or ‘extracellular vesicle’ that has intercellular signaling functions, and thus studies on the components and functions thereof have been actively conducted.

Cells shed various membranous vesicles to their extracellular environment, and these released vesicles are usually called extracellular vesicles (EVs). The EV is also called cell membrane-derived vesicle, ectosome, shedding vesicle, microparticle, exosome, etc., and is also used discriminately from exosome in some cases.

Exosome is a vesicle of tens to hundreds of nanometers in size, which comprises a phospholipid bilayer membrane having the same structure as that of the cell membrane. This exosome contains proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosome is an intercellular signaling mediator secreted by cells, and various cellular signals transmitted through it regulate cellular behaviors, including the activation, growth, migration, differentiation, dedifferentiation, apoptosis, and necrosis of target cells. Exosome contains specific genetic materials and bioactive factors depending on the nature and state of cells from which the exosome was derived. Exosome derived from proliferating stem cells regulates cell behaviors such as cell migration, proliferation and differentiation, and recapitulates the characteristics of stem cells involved in tissue regeneration (Nature Review Immunology 2002 (2) 569-579).

That is, exosomes called “avatars” of cells contain bioactive factors such as growth factors, similar to cells, and serve as carriers that transmit bioactive factors between cells, that is, serve to mediate cell-to-cell communication. Exosomes are known to be released not only from animal cells such as stem cells, immune cells, fibroblasts and cancer cells, but also from cells of various organisms such as plants, bacteria, fungi, and algae.

Conventional techniques for isolating exosomes include ultracentrifugation, density gradient centrifugation, ultrafiltration, tangential flow filtration (TFF), size exclusion chromatography, ion-exchange chromatography, immunoaffinity capture, microfluidics-based isolation, exosome precipitation, total exosome isolation kit, polymer based precipitation, and the like.

However, although various methods for isolating exosomes as described above have been proposed, continuous improvement can be made in the technical field related to the production of exosomes, similar to other technical fields. For example, there is a need to provide a new technique for producing exosomes, which can enhance the productivity of exosomes per cell-derived conditioned medium and improve the efficacy related to bioactive factors of exosomes.

Meanwhile, it is to be understood that the matters described as the background art are intended merely to aid in the understanding of the background of the present invention and are not admitted as prior art against the present invention.

### SUMMARY

An object of the present invention is to provide a method for producing exosomes, which can increase exosome production and/or enhance bioactivity of exosomes.

Another object of the present invention is to provide a method for increasing exosome production and a method for enhancing bioactivity of exosomes.

Still another of the present invention is to provide a medium for increasing exosome production and/or enhancing bioactivity of exosomes.

Yet another object of the present invention is to provide various applications of exosomes having enhanced bioactivity, obtained by the production method as described above.

However, the objects of the present invention as described above are illustrative and the scope of the present invention is not limited thereby. In addition, other objects and advantages of the present invention will be more apparent from the following description, the appended claims and the accompanying drawings.

### DISCLOSURE

The present inventors have conducted intensive studies on a method for producing exosomes, which can increase exosome production and enhance efficacy of exosomes, and as a result, have found that, when a combination of TNF-α and interferon-γ is used, the amount of exosomes produced per conditioned medium (or per cell) increases and the bioactivity of produced exosomes is enhanced, thereby completing the present invention.

The present invention provides a method for producing exosomes, the method comprising culturing animal cells in a medium containing TNF-α and interferon-γ.

As used herein, the term “extracellular vesicles (EVs)” is generally meant to encompass membrane vesicles, ectosomes, shedding vesicles, microparticles, or equivalents thereto. Depending on the isolation environment, conditions and methods, the term “extracellular vesicles” may have the same meaning as the term “exosomes”, and may also be meant to include nanovesicles that have the same or similar size as exosomes, but have a composition which differs from that of exosomes. Meanwhile, the term “exosomes” as used in relation to the productivity is meant to encompass the aforesaid extracellular vesicles.

As used herein, the term “exosomes” refers to vesicles of tens to hundreds of nanometers in size (preferably, about 30 to 200 nm), which comprise a phospholipid bilayer membrane having the same structure as that of the cell membrane (however, the particle size of exosomes is variable depending on the type of cell from which the exosomes are isolated, an isolation method and a measurement method) (Vasiliy S. Chernyshev et al., “Size and shape characterization of hydrated and desiccated exosomes”, Anal Bioanal Chem, (2015) DOI 10.1007/s00216-015-8535-3). These exosomes contain proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosomes are intercellular signaling mediators secreted by cells, and various cellular signals transmitted through them regulate cellular behaviors, including the activation, growth, migration, differentiation, dedifferentiation, apoptosis, and necrosis of target cells.

Meanwhile, the term “exosomes” as used herein is intended to include all vesicles (e.g., exosome-like vesicles) which are secreted from animal cells and released into extracellular spaces, and have a nano-sized vesicle structure and a composition similar to that of exosomes.

In the present invention, the type of animal cells from which exosomes are derived is not limited. As an example not limiting the scope of the present invention, the animal cells may be stem cells or immune cells. The stem cells may be embryonic stem cells, induced pluripotent stem cells (iPSCs), adult stem cells, embryonic stem cell-derived mesenchymal stem cells, or induced pluripotent stem cell-derived mesenchymal stem cells. The immune cells may be T cells, B cells, NK cells, cytotoxic T cells, dendritic cells or macrophages.

As an example not limiting the scope of the present invention, the adult stem cells may be at least one type of adult stem cells selected from the group consisting of mesenchymal stem cells, human tissue-derived mesenchymal stromal cells, human tissue-derived mesenchymal stem cells, and pluripotent stem cells. The mesenchymal stem cells may be mesenchymal stem cells derived from at least one tissue selected from the group consisting of umbilical cord, umbilical cord blood, bone marrow, adipose tissue, muscle, nerve, skin, amniotic membrane, Wharton's jelly, and placenta. Preferably, the adult stem cells may be mesenchymal stem cells, for example, adipose-, bone marrow-, umbilical cord- or umbilical cord blood-derived stem cells, more preferably adipose-derived stem cells. The stem cells or the immune cells are not limited to the kind thereof, as long as they do not pose a risk of infection with a pathogen and do not cause immune rejection, but may preferably be human stem cells or human immune cells.

However, it is of course possible to use various animal cells that are being used in the art or may be used in the future, as long as they do not cause adverse effects on the human body. For example, it is also possible to use HEK293 cells or HEK293T cells. Thus, it is to be understood that the adipose-derived stem cell used in the Examples described later is an example of animal cells that may be used in the present invention, and the present invention is not limited thereto.

As used herein, the term “pre-treated” refers to culturing animal cells in a medium containing TNF-α and interferon-γ, and the term “pre-treated exosomes” refers to exosomes obtained by culturing animal cells in a medium containing TNF-α and interferon-γ. The term “untreated” means culturing animal cells in a medium that does not contain TNF-α and interferon-γ, and the term “untreated exosomes” or “untreated control” refers to exosomes obtained without being treated with TNF-α and interferon-γ in any of a cell culture step and an exosome production step.

As used herein, the term “serum-free medium” or “SFM” refers to, for example, a culture medium (optionally containing antibiotics and/or antifungal agents) comprising a basal medium not supplemented with serum such as fetal bovine serum (FBS), and the term “EDM” refers to a culture medium (optionally containing antibiotics and/or antifungal agents) comprising a basal medium supplemented with a platelet lysate, preferably a human platelet lysate, more preferably a human platelet lysate from which platelet lysate-derived exosomes and extracellular vesicles have been removed. As an example not limiting the scope of the present invention, the basal medium may be α-MEM, DMEM, DMEM F12, 199 medium, RPMI-1640, L-15, Hum F-10, Hum F-12, DM-160, DM-170, or the like.

The present invention provides a method for increasing exosome production, the method comprising culturing animal cells in a medium containing TNF-α and interferon-γ. For example, the method for increasing exosome production according to one embodiment of the present invention may comprise steps of: (a) culturing animal cells in a medium containing TNF-α and interferon-γ; (b) washing the animal cells; (c) culturing the animal cells in a serum-free medium and collecting a conditioned medium of the animal cells; and (d) isolating exosomes from the collected conditioned medium.

In the method for increasing exosome production according to one embodiment of the present invention, the serum-free medium in step (c) may be supplemented with a platelet lysate. The platelet lysate may be preferably a human platelet lysate, more preferably a human platelet lysate from which platelet lysate-derived exosomes and extracellular vesicles have been removed.

In the method for increasing exosome production according to one embodiment of the present invention, each of TNF-α and interferon-γ may be contained in the medium at a concentration of about 1 ng/mL to 100 ng/mL, preferably about 10 ng/mL to 30 ng/mL, for example, about 20 ng/mL.

In the method for increasing exosome production according to one embodiment of the present invention, the culturing may be performed for about 12 hours to 48 hours, for example, 24 hours.

In the method for increasing exosome production according to one embodiment of the present invention, the exosome production may be defined as the number of exosome particles per mL of the conditioned medium (or the number of exosome particles per cell).

The present invention provides a method for enhancing bioactivity of exosomes, the method comprising culturing animal cells in a medium containing TNF-α and interferon-γ. For example, the method for enhancing bioactivity of exosomes according to one embodiment of the present invention may comprise steps of: (a) culturing animal cells in a medium containing TNF-α and interferon-γ; (b) washing the animal cells; (c) culturing the animal cells in a serum-free medium and collecting a conditioned medium of the animal cells; and (d) isolating exosomes from the collected conditioned medium.

In the method for enhancing bioactivity of exosomes according to one embodiment of the present invention, the serum-free medium in step (c) may be supplemented with a platelet lysate. The platelet lysate may be preferably a human platelet lysate, more preferably a human platelet lysate from which platelet lysate-derived exosomes and extracellular vesicles have been removed.

In the method for enhancing bioactivity of exosomes according to one embodiment of the present invention, each of TNF-α and interferon-γ may be contained in the medium at a concentration of about 1 ng/mL to 100 ng/mL, preferably about 10 ng/mL to 30 ng/mL, for example, about 20 ng/mL.

In the method for enhancing bioactivity of exosomes according to one embodiment of the present invention, the culturing may be performed for about 12 hours to 48 hours, for example, 24 hours.

In the method for enhancing bioactivity of exosomes according to one embodiment of the present invention, the enhancement of bioactivity of exosomes may be enhancement in increasing elastin synthesis of exosomes, for example, enhancement of at least one of a skin elasticity improvement effect and a wrinkle reduction effect.

The present invention provides a medium for increasing production of exosomes derived from animal cells or enhancing bioactivity of exosomes derived from animal cells.

In the medium for increasing production of exosomes derived from animal cells or enhancing bioactivity of exosomes derived from animal cells according to one embodiment of the present invention, each of TNF-α and interferon-γ may be contained in the medium at a concentration of about 1 ng/mL to 100 ng/mL, preferably about 10 ng/mL to 30 ng/mL, for example, about 20 ng/mL.

The present invention also provides a composition containing, as an active ingredient, exosomes having an enhanced effect of increasing elastin synthesis, which are obtained by culturing animal cells in a medium containing TNF-α and interferon-γ. The composition may be, for example, a composition for skin elasticity improvement or wrinkle reduction.

For example, the composition according to one embodiment of the present invention may be obtained by performing steps of: (a) culturing animal cells in a medium containing TNF-α and interferon-γ; (b) washing the animal cells; (c) culturing the animal cells in a serum-free medium and collecting a conditioned medium of the animal cells; and (d) isolating exosomes from the collected conditioned medium.

In the composition according to one embodiment of the present invention, the serum-free medium in step (c) may be supplemented with a platelet lysate. The platelet lysate may be preferably a human platelet lysate, more preferably a human platelet lysate from which platelet lysate-derived exosomes and extracellular vesicles have been removed.

In the composition according to one embodiment of the present invention, each of TNF-α and interferon-γ may be contained in the medium at a concentration of about 1 ng/mL to 100 ng/mL, preferably about 10 ng/mL to 30 ng/mL, for example, about 20 ng/mL.

In the composition according to one embodiment of the present invention, the culturing may be performed for about 12 hours to 48 hours, for example, 24 hours.

As an example not limiting the scope of the present invention, the composition containing exosomes obtained according to the method for producing exosomes may be prepared as a quasi-drug, a cosmetic composition or a skin external preparation. The quasi-drug or the skin external preparation may be prepared as solutions, ointments, creams, sprays, patches, gels, or aerosols. The cosmetic composition according to one embodiment of the present invention may be, for example, cream or lotion.

Meanwhile, when the composition according to one embodiment of the present invention is prepared as a quasi-drug, a skin external preparation and/or a cosmetic composition, it may suitably contain components which are generally used in quasi-drugs, skin external preparations and/or cosmetic compositions, for example, moisturizers, antioxidants, oily components, UV absorbers, emulsifiers, surfactants, thickeners, alcohols, powder components, colorants, aqueous components, water, and various skin nutrients, etc., as needed, within the range that does not impair the effect of the present invention.

Furthermore, the quasi-drug, the skin external preparation and/or the cosmetic composition according to one embodiment of the present invention may comprise, in addition to the exosomes having enhanced bioactivity, a conventional skin elasticity improver, anti-wrinkle agent and/or moisturizing agent within a range that does not impair the effect (e.g., skin elasticity improvement effect and/or wrinkle reduction effect) thereof. For example, the composition comprising the exosomes having enhanced bioactivity as active ingredients according to the present invention may be contained in or mixed with at least one of hydrogel, hyaluronic acid, salt of hyaluronic acid (e.g., sodium hyaluronate, etc.), or hyaluronate gel. In the quasi-drug, the skin external preparation and/or the cosmetic composition according to one embodiment of the present invention, the kind of hydrogel is not particularly limited, but the hydrogel may be preferably obtained by dispersing a gelled polymer in a polyhydric alcohol. The gelled polymer may be at least one selected from the group consisting of pluronic, purified agar, agarose, gellan gum, alginic acid, carrageenan, cassia gum, xanthan gum, galactomannan, glucomannan, pectin, cellulose, guar gum, and locust bean gum, and the polyhydric alcohol may be at least one selected from the group consisting of ethylene glycol, propylene glycol, 1,3-butylene glycol, isobutylene glycol, dipropylene glycol, sorbitol, xylitol, and glycerin.

The quasi-drug, the skin external preparation and/or the cosmetic composition according to one embodiment of the present invention may be used in various forms, for example, patches, mask packs, mask sheets, creams, tonics, ointments, suspensions, emulsions, pastes, lotions, gels, oils, packs, sprays, aerosols, mists, foundations, powders, and oilpapers. For example, the quasi-drug, the skin external preparation and/or the cosmetic composition may be applied to or soaked in at least one surface of a patch, a mask pack or a mask sheet.

The cosmetic composition according to one embodiment of the present invention is used for the purpose of skin elasticity improvement effect and/or wrinkle reduction, etc., and the cosmetic composition may be prepared as any formulation which is generally prepared in the art. For example, it may be formulated as patch, mask pack, mask sheet, skin softener, nutrition, astringent lotion, nourishing cream, massage cream, eye cream, cleansing cream, essence, eye essence, cleansing lotion, cleansing foam, cleansing water, sunscreen, lipstick, soap, shampoo, surfactant-containing cleanser, bath preparation, body lotion, body cream, body oil, body essence, body cleanser, hairdye, hair tonic, etc., but is not limited thereto.

The quasi-drug, the skin external preparation and/or the cosmetic composition according to one embodiment of the present invention contains components which are commonly used in quasi-drugs, skin external preparations and/or cosmetic products. For example, the quasi-drug, the skin external preparation and/or the cosmetic composition may contain conventional adjuvants and carriers, such as antioxidants, stabilizers, solubilizers, vitamins, pigments, and fragrances. In addition, other components in each formulation for the quasi-drug, the skin external preparation and/or the cosmetic composition may be suitably selected without difficulty by those skilled in the art depending on the type or intended use of the quasi-drug, the skin external preparation and/or the cosmetic composition.

### Advantageous Effects

According to the present invention, it is possible not only to increase production of exosomes to be isolated from a cell-derived conditioned medium, but also to exhibit an excellent effect of enhancing bioactivity of exosomes, such as increasing elastin synthesis of exosomes. Therefore, the method for producing exosomes according to the present invention has advantages that it can economically and efficiently produce exosomes having enhanced bioactivity, which can be utilized commercially and/or clinically, in high yield, and in particular, it can produce a large amount of exosomes having enhanced bioactivity which is much better than that of exosomes produced by a conventional method.

It should be understood that the scope of the present invention is not limited to the aforementioned effects.

### BRIEF DESCRIPTION OF DRAWINGS

FIG. 1A depicts a graph showing the particle size distribution and the number of particles obtained by performing nanoparticle tracking analysis (NTA) for untreated exosomes obtained using ultrafiltration after culturing stem cells in a medium not containing TNF-α and interferon-γ.

FIG. 1B depicts a graph showing the particle size distribution and the number of particles obtained by performing nanoparticle tracking analysis (NTA) for pre-treated exosomes obtained using ultrafiltration after culturing stem cells in a medium containing TNF-α and interferon-γ.

FIG. 2A and FIG. 2B depict comparative graphs showing that exosome production in the case of pre-treatment with TNF-α and interferon-γ according to one embodiment of the present invention remarkably increased as compared with a control group not treated with TNF-α and interferon-γ. FIG. 2A is a graph showing the results of measuring and comparing the numbers of exosome particles in conditioned media (CM) before performing ultrafiltration on the conditioned media (CM); FIG. 2B a graph showing the results of measuring and comparing the numbers of exosome particles in exosome solutions obtained after performing ultrafiltration on the conditioned media (CM); and FIG. 2C is a graph showing the comparison results of FIGS. 2A and 2B together.

FIG. 3A depicts a graph showing the particle size distribution and the number of particles obtained by performing nanoparticle tracking analysis (NTA) for untreated exosomes obtained using ultracentrifugation after culturing stem cells in a medium not containing TNF-α and interferon-γ.

FIG. 3B depicts a graph showing the particle size distribution and the number of particles obtained by performing nanoparticle tracking analysis (NTA) for pre-treated exosomes obtained using ultracentrifugation after culturing stem cells in a medium containing TNF-α and interferon-γ.

FIG. 4A to FIG. 4C depict comparative graphs showing that exosome production in the case of pre-treatment with TNF-α and interferon-γ according to one embodiment of the present invention remarkably increased as compared with a control group not treated with TNF-α and interferon-γ. FIG. 4A is a graph showing the results of measuring and comparing the numbers of exosome particles in conditioned media (CM) before performing ultracentrifugation on the conditioned media (CM); FIG. 4B a graph showing the results of measuring and comparing the numbers of exosome particles in exosome solutions (EXO) obtained after performing ultracentrifugation on the conditioned media (CM); and FIG. 4C is a graph showing the comparison results of FIGS. 4A and 4B together.

FIG. 5 is a comparative graph showing that the content of a CD63 marker (as an exosome specific marker, reflecting the content and productivity of exosomes) in an experimental group pre-treated with TNF-α and interferon-γ according to one embodiment of the present invention remarkably increased compared to the content of the CD63 marker in each of an untreated control group, a group pre-treated with interferon-γ alone and a group pre-treated with TNF-α alone.

FIG. 6 is a comparative graph showing that when human dermal fibroblasts were treated with pre-treated exosomes according to one embodiment of the present invention, elastin synthesis in the human dermal fibroblasts remarkably increased as compared with a control group not-treated with any exosomes, and that when human dermal fibroblasts were treated with pre-treated exosomes according to one embodiment of the present invention, the increase of elastin synthesis in the human dermal fibroblasts was remarkably superior to that of each of untreated exosomes (common exosomes), exosomes pre-treated with interferon-γ alone, and exosomes pre-treated with TNF-α alone.

FIG. 7 is a schematic view showing during cell culture, a process of pre-treating cells with TNF-α and interferon-γ in a cell growth step, and after washing the pre-treated cells, a process of collecting conditioned media containing exosomes secreted by culturing the cells in SFM medium or EDM medium, in order to produce exosomes having enhanced bioactivity according to one embodiment of the present invention.

FIG. 8 shows that the exosomes produced in EDM medium after pre-treatment with TNF-α and interferon-γ according to one embodiment of the present invention remarkably increased elastin synthesis in human dermal fibroblasts compared to the exosomes produced in SFM medium after pre-treatment with TNF-α and interferon-γ.

### EXAMPLES

Hereinafter, the present invention will be described in more detail with reference to the following examples. However, the following examples are only to illustrate the present invention and are not intended to limit or restrict the scope of the present invention. Those that can be easily inferred by those skilled in the art from the detailed description and examples of the present invention are interpreted as falling within the scope of the present invention. References referred to in the present invention are incorporated herein by reference.

Throughout the present specification, it is to be understood that, when any part is referred to as “comprising” any component, it does not exclude other components, but may further include other components, unless otherwise specified.

### Example 1: Cell Culture

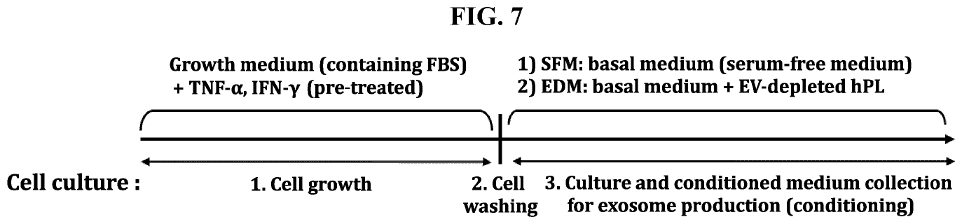

According to a cell culture method known in the art to which the present invention pertains, adipose-derived stem cells were cultured in MEM-α (Minimum Essential Medium alpha; purchased from ThermoFisher Scientific) supplemented with 10% FBS (Fetal Bovine Serum; purchased from ThermoFisher Scientific) and 1% penicillin-streptomycin (purchased from ThermoFisher Scientific) at 37° C. under 5% CO₂. Where stem cells were not treated with TNF-α and interferon-γ (IFN-γ), the stem cells were maintained in culture for 24 hours. Where stem cells were treated with TNF-α and interferon-γ (IFN-γ), the medium was treated with TNF-α and IFN-γ at concentrations of 20 ng/mL, respectively, and the stem cells were cultured in the medium for 24 hours (see “1. Cell growth” in FIG. 7). After 24 hours of culture, both the stem cells treated with TNF-α and IFN-γ and the stem cells not treated with TNF-α and IFN-γ were washed with phosphate-buffered saline (“2. Cell washing” in FIG. 7). Then, the media were replaced with serum-free medium (SFM) or EDM, the stem cells were cultured for 24 hours, and each culture supernatant (hereinafter referred to as conditioned medium) was collected (see “3. Culture and conditioned medium collection for exosome production” in FIG. 7). Meanwhile, EDM may be prepared by collecting a permeate obtained after tangential flow filtration of human platelet lysate (hPL), thus obtaining a human platelet lysate from which extracellular vesicles and exosomes have been removed, and adding the obtained platelet lysate to a basal medium.

Examples 2 to 5 as described later will explain exosomes produced in a serum-free medium after being pre-treated with TNF-α and interferon-γ, and Example 6 will explain that exosomes produced in EDM after being pre-treated with TNF-α and interferon-γ have a superior effect in increasing elastin synthesis to exosomes produced in SFM after being pre-treated with TNF-α and interferon-γ.

### Example 2: Isolation and Purification of Exosomes

### Example 2-1: Isolation Using Ultrafiltration

Ultrafiltration was used for isolation and concentration of exosomes from each of the conditioned media collected in Example 1. A centrifugal filter (purchased from Millipore) was used as a filter for ultrafiltration. Centrifugal filters may be selected with various molecular weight cutoffs (MWCOs). Using the filter having selected MWCO, exosomes were isolated and concentrated, and particles, proteins, lipids, nucleic acids, low-molecular-weight compounds, etc., were removed, which are smaller than the MWCO.

A centrifugal filter having a MWCO of 30,000 Da (Dalton) was used to isolate and concentrate exosomes. Using ultrafiltration, exosomes were isolated from the conditioned medium by removing substances smaller than the MWCO and concentrating the conditioned medium to a volume of about 1/200.

Meanwhile, as methods of isolating exosomes from a conditioned medium of animal cells including stem cells, various methods known in the art may be used in addition to the isolation method as described above. For example, for isolation of exosomes, known isolation methods may be used, such as ultracentrifugation, density gradient centrifugation, tangential flow filtration (TFF), size exclusion chromatography, ion exchange chromatography, immunoaffinity capture, microfluidics-based isolation, exosome precipitation, total exosome isolation kit, polymer based precipitation and the like. However, the method for isolating exosomes is not limited to the above-described methods, and it is of course possible to use various isolation methods that are being used in the art or may be used in the future.

### Example 2-2: Isolation of Exosomes Using Ultracentrifugation

In addition, ultracentrifugation was used for isolation and concentration of exosomes from each of the conditioned media collected in Example 1. The collected conditioned medium was subjected to sequential centrifugation at 4° C. to isolate exosomes as follows. First, to remove cells from the collected conditioned medium, the conditioned medium was centrifuged at 300×g for 10 minutes, and then the supernatant was collected. In addition, to remove cell debris from the supernatant, the supernatant was centrifuged at 2,000×g for 20 minutes, and then the supernatant was collected. Then, to remove microvesicles from the supernatant, the supernatant was centrifuged at 16,500×g for 10 minutes, and then the supernatant was collected. The finally obtained supernatant was centrifuged at 120,000×g for 120 minutes, and then the supernatant was discarded and the pellets were collected. Then, the pellets were washed with phosphate-buffered saline, and centrifuged again at 120,000×g for 120 minutes, and the supernatant was discarded. Next, the pellets were suspended in phosphate-buffered saline, thereby isolating exosomes.

### Example 3: Characterization of Isolated Exosomes and Evaluation of Exosome Productivity

### Example 3-1: Characterization and Evaluation of Productivity of Exosomes Isolated Using Ultrafiltration

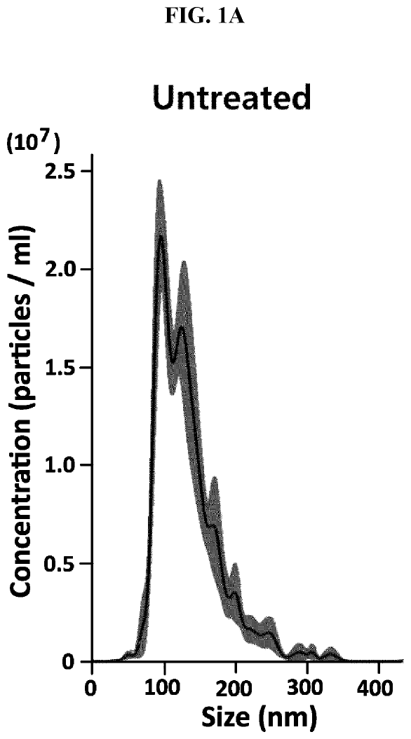

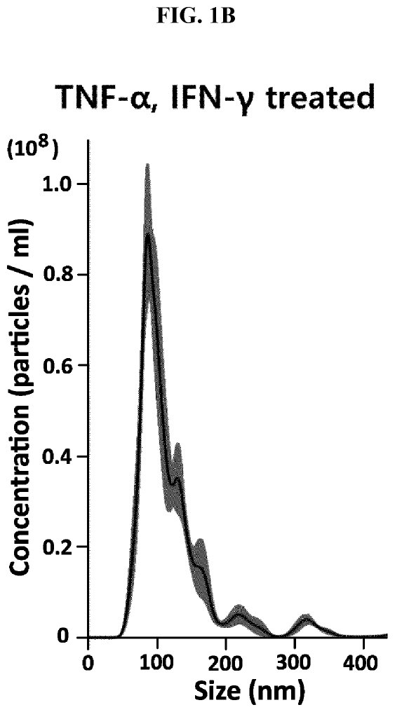

The particle size and concentration of the exosomes isolated according to Example 2-1 were measured by nanoparticle tracking analysis (NTA) instrument (purchased from Malvern). FIGS. 1A and 1B depict graphs showing the results of NTA of the exosomes isolated according to Example 2-1. As shown in FIGS. 1A and 1B, it could be seen that the pre-treated exosomes (indicated with “TNF-α, IFN-γ treated” in FIG. 1B) obtained using ultrafiltration after culturing the stem cells in the medium containing TNF-α and interferon-γ according to one embodiment of the present invention had a more uniform particle size distribution than the untreated exosomes (indicated with “Untreated” in FIG. 1A) obtained using ultrafiltration after culturing the stem cells in the medium not containing TNF-α and interferon-γ.

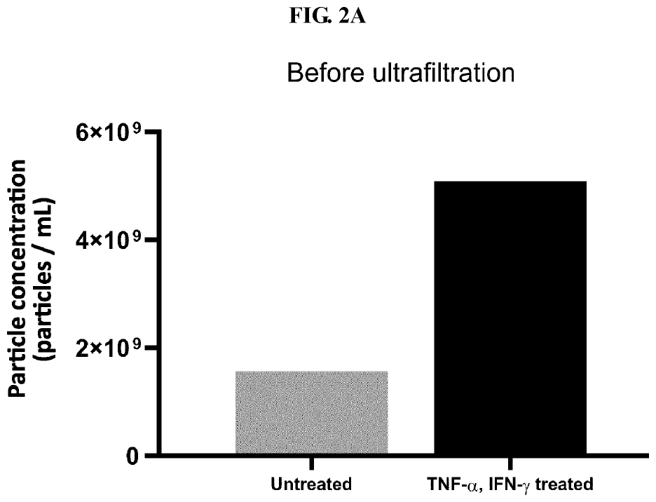

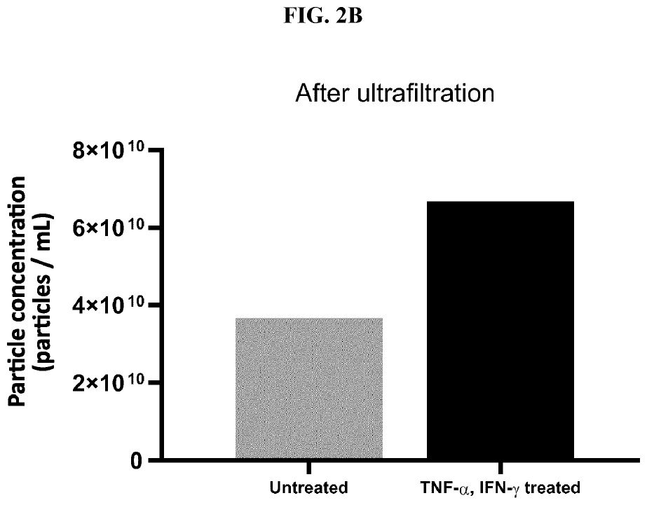

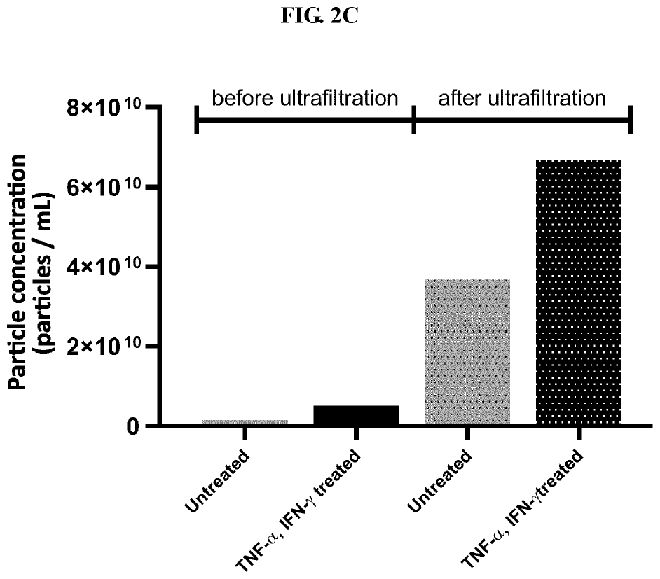

In addition, as shown in FIGS. 2A, 2B and 2C, from comparative analysis results of NTA, it was confirmed that the exosome productivity in the case of pre-treatment with TNF-α and interferon-γ (indicated with “TNF-α, IFN-γ treated” in FIGS. 2A, 2B and 2C) increased by approximately 2 to 3 times or more as compared with the control group not treated with TNF-α and interferon-γ (indicated with “Untreated” in FIGS. 2A, 2B and 2C).

As a result of measuring and comparing the numbers of exosome particles in conditioned media (CM) before performing ultrafiltration on the conditioned media (CM), it was confirmed that the exosome productivity in the case of pre-treatment with TNF-α and interferon-γ remarkably increased as compared with the control group not treated with TNF-α and interferon-γ (FIGS. 2A and 2C). In addition, as a result of measuring and comparing the numbers of exosome particles in the exosome solutions obtained after performing ultrafiltration on the conditioned media (CM), it was confirmed that the exosome productivity in the case of pre-treatment with TNF-α and interferon-γ remarkably increased as compared with the control group not treated with TNF-α and interferon-γ (FIGS. 2B and 2C).

Therefore, it can be seen that the present invention can economically and efficiently produce exosomes having a uniform particle size distribution in high yield by performing a pre-treatment process of culturing stem cells in a medium containing a combination of TNF-α and interferon-γ.

### Example 3-2: Characterization and Evaluation of Productivity of Exosomes Isolated Using Ultracentrifugation

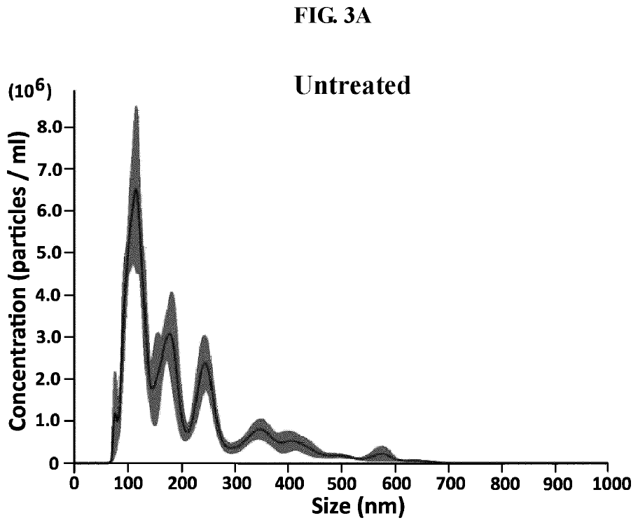

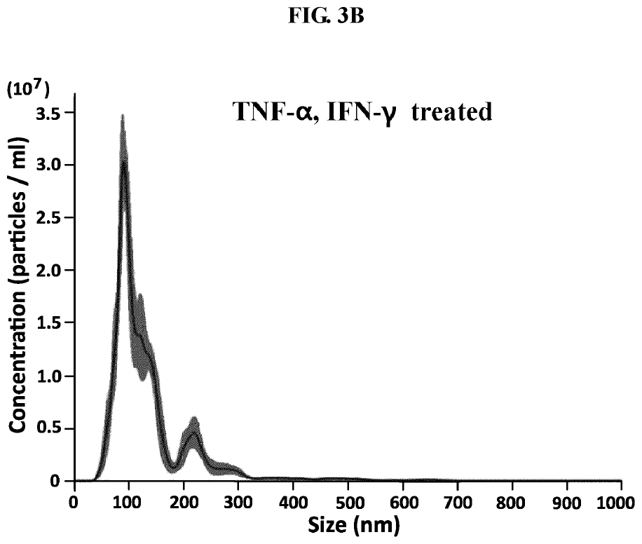

The particle size and concentration of the exosomes isolated according to Example 2-2 were measured by nanoparticle tracking analysis (NTA) instrument (purchased from Malvern). FIGS. 3A and 3B depict graphs showing the results of NTA of the exosomes isolated according to Example 2-2. As shown in FIGS. 3A and 3B, it could be seen that the pre-treated exosomes (indicated with “TNF-α, IFN-γ treated” in FIG. 3B) obtained using ultracentrifugation after culturing the stem cells in the medium containing TNF-α and interferon-γ according to one embodiment of the present invention had a more uniform particle size distribution than the untreated exosomes (indicated with “Untreated” in FIG. 3A) obtained using ultracentrifugation after culturing the stem cells in the medium not containing TNF-α and interferon-γ.

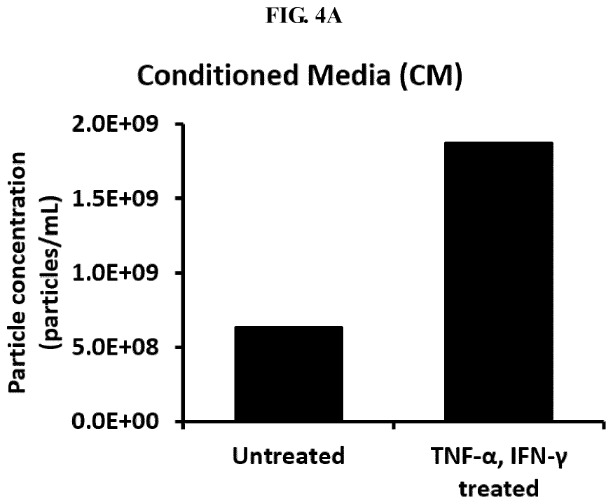

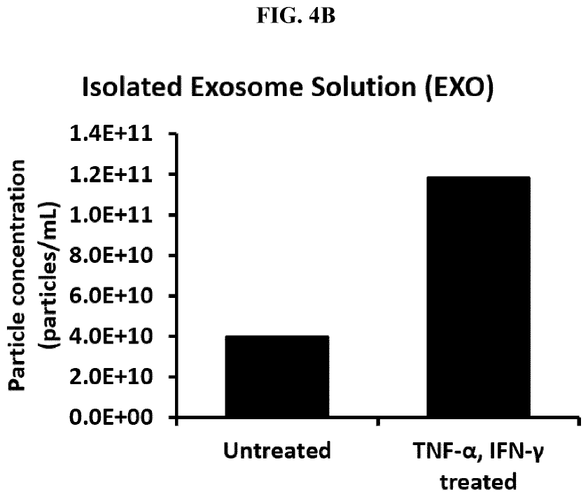

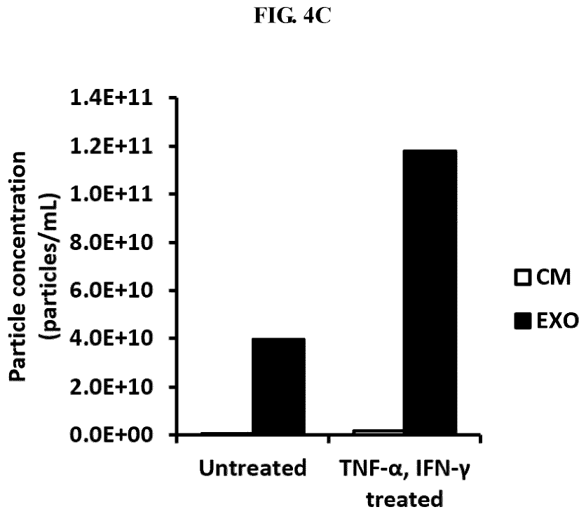

In addition, as shown in FIGS. 4A, 4B and 4C, from comparative analysis results of NTA, it was confirmed that the exosome productivity in the case of pre-treatment with TNF-α and interferon-γ (indicated with “TNF-α, IFN-γ treated” in FIGS. 4A, 4B and 4C) increased by approximately 2 to 3 times or more as compared with the control group not treated with TNF-α and interferon-γ (indicated with “Untreated” in FIGS. 4A, 4B and 4C).

As a result of measuring and comparing the numbers of exosome particles in conditioned media (CM) before performing ultracentrifugation on the conditioned media (CM), it was confirmed that the exosome productivity in the case of pre-treatment with TNF-α and interferon-γ remarkably increased as compared with the control group not treated with TNF-α and interferon-γ (FIGS. 4A and 4C). In addition, as a result of measuring and comparing the numbers of exosome particles in the exosome solutions (EXO) obtained after performing ultracentrifugation on the conditioned media (CM), it was confirmed that the exosome productivity in the case of pre-treatment with TNF-α and interferon-γ remarkably increased as compared with the control group not treated with TNF-α and interferon-γ (FIGS. 4B and 4C).

Therefore, it can be seen that the present invention can economically and efficiently produce exosomes having a uniform particle size distribution in high yield by performing the pre-treatment process of culturing stem cells in a medium containing a combination of TNF-α and interferon-γ.

### Example 4: Analysis as to Whether Exosome Productivity is Increased by Pre-Treatment Process of Culturing Stem Cells in Medium Containing Combination of TNF-α and Interferon-γ

In addition, for accurate comparative analysis of exosome productivity, experimental groups were established as follows according to whether TNF-α and/or interferon-γ were/was included in stem cell culture for exosome production:

(1) Untreated control group (untreated): an experimental group in which stem cells were cultured in a medium not containing TNF-α and interferon-γ, and then exosomes were isolated according to Example 2-2;

(2) group pre-treated with interferon-γ alone (IFN-γ treated): an experimental group in which stem cells were cultured in a medium containing interferon-γ, and then exosomes were isolated according to Example 2-2;

(3) group pre-treated with TNF-α alone (TNF-α treated): an experimental group in which stem cells were cultured in a medium containing TNF-α, and then exosomes were isolated according to Example 2-2; and

(4) group pre-treated with TNF-α and interferon-γ (TNF-α, IFN-γ treated): an experimental group in which stem cells were cultured in a medium containing TNF-α and interferon-γ, and then exosomes were isolated according to Example 2-2.

The exosomes of each of experimental groups (1), (2), (3) and (4) and Exosome-Human CD63 Flow Detection Reagent (Invitrogen™) were mixed together overnight. Then, each of the mixtures was allowed to react with PE mouse anti-Human CD63 (BD Parmigen™), and the PE mean fluorescence intensity (MFI) thereof was measured using flow cytometry.

Meanwhile, since CD63 is a representative positive marker of exosomes, the CD63 content and the exosome content are linearly proportional to each other, and an increase in the CD63 content means a proportional increase in the exosome content. According to this principle, human CD63 protein of known concentration was serially diluted to prepare human CD63 protein solutions having different human CD63 protein concentrations, and each human CD63 protein solution and Exosome-Human CD63 Flow Detection Reagent (Invitrogen™) were mixed together overnight. Then, each of the mixtures was allowed to react with PE mouse anti-human CD63 (BD Parmigen™), and the PE mean fluorescence intensity (MFI) thereof was measured using flow cytometry. Then, linear regression analysis was performed on the human CD63 protein concentration values and the corresponding MFI measurement values, and a standard quantitative analysis graph satisfying a linearity of 0.99 or higher was generated. Then, using the generated standard quantitative analysis graph, the CD63 content in each of experimental groups (1), (2), (3) and (4) was determined.

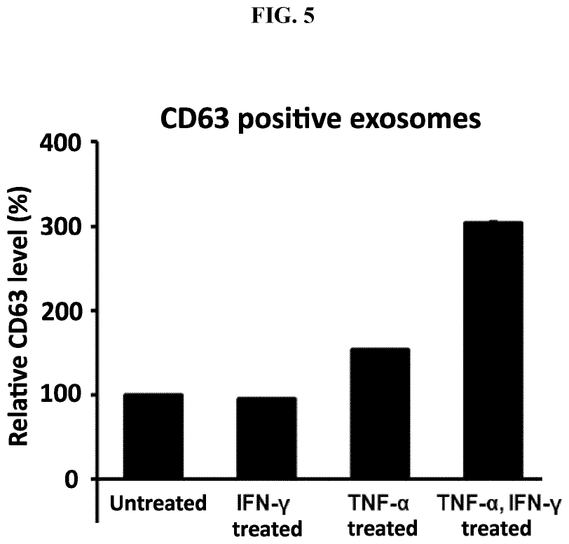

As a result, it could be confirmed that the exosome content (CD63 content) measured in experimental group (4) pre-treated with TNF-α and interferon-γ according to the present invention was remarkably higher than the exosome content measured in each of experimental group (1) untreated, and experimental group (2) pre-treated with interferon-γ alone, and experimental group (3) pre-treated with TNF-α alone (see FIG. 5). Therefore, it can be seen that the present invention can dramatically increase the exosome productivity by performing the pre-treatment process of culturing stem cells in a medium containing a combination of TNF-α and interferon-γ.

### Example 5: Evaluation 1 of Effect of Pre-Treated Exosomes on Increase in Elastic Synthesis

Using human dermal fibroblasts (HDFs), the effect of stem cell-derived exosomes pre-treated with TNF-α and/or interferon-γ (IFN-γ), on an increase in elastin synthesis, was evaluated as follows. To this end, experimental groups were established as follows:

(1) Negative control group (control): an experimental group in which human dermal fibroblasts were not treated with exosomes;

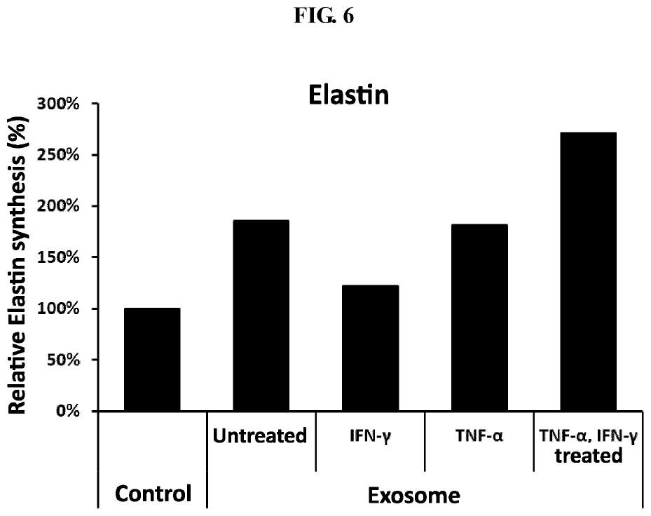

(2) group treated with untreated exosomes (common exosomes): an experimental group in which human dermal fibroblasts were treated with the common exosomes of experimental group (1) of Example 4 at a concentration of 2.26×10¹⁰ particles/mL (indicated with “Untreated” in FIG. 6);

(3) group treated with exosomes pre-treated with IFN-γ alone: an experimental group in which human dermal fibroblasts were treated with the exosomes of experimental group (2) of Example 4 at a concentration of 2.26×10¹⁰ particles/mL (indicated with “IFN-γ” in FIG. 6);

(4) group treated with exosomes pre-treated with TNF-α alone: an experimental group in which human dermal fibroblasts were treated with the exosomes of experimental group (3) of Example 4 at a concentration of 2.26×10¹⁰ particles/mL (indicated with “TNF-α” in FIG. 6); and

(5) group treated with exosomes pre-treated with TNF-α and IFN-γ: an experimental group in which human dermal fibroblasts were treated with the exosomes (pre-treated with TNF-α and IFN-γ) of experimental group (4) of Example 4 at a concentration of 2.26×10¹⁰ particles/mL (indicated with “TNF-α, IFN-γ treated” in FIG. 6).

Human dermal fibroblasts were suspended in EDF growth medium (purchased from CEFO Co., Ltd., Seoul, Korea) containing 10% growth supplement and 1% penicillin-streptomycin, and then seeded into a 48-well plate at a density of 3.0×10⁴ cells/well and cultured in an incubator at 37° C. under 5% CO₂ for 24 hours.

Thereafter, the human dermal fibroblasts were treated according to the condition of each of experimental groups (1) to (5) above, and the human dermal fibroblasts were cultured in an incubator at 37° C. under 5% CO₂ for 72 hours. After 72 hours, the culture supernatant of the human dermal fibroblasts was collected, and the amount of elastin synthesis in the culture supernatant was measured using an elastin ELISA kit (purchased from CUSABIO). The measured amount of elastin synthesis was normalized by the final number of human dermal fibroblasts measured with an MTT assay kit (purchased from Sigma).

As shown in FIG. 6, the elastin synthesis in experimental group (5), in which human dermal fibroblasts were treated with the exosomes pre-treated with TNF-α and IFN-γ according to the present invention, remarkably increased compared to that in each of experimental group (2) in which human dermal fibroblasts were treated with common exosomes, experimental group (3) in which human dermal fibroblasts were treated with the exosomes pre-treated with IFN-γ alone, and experimental group (4) in which human dermal fibroblasts were treated with the exosomes pre-treated with TNF-α alone.

Therefore, it can be seen that the present invention can dramatically enhance the effect of exosomes on an increase in elastin synthesis by performing the pre-treatment process of culturing stem cells in a medium containing a combination of TNF-α and interferon-γ.

These results show that the method for producing exosomes according to the present invention is able to not only improve the productivity of exosomes to be isolated from a cell-derived conditioned medium, but also is excellent in terms of enhancing bioactivity of exosomes, that is, in terms of enhancing the effect of exosomes on an increase in elastin synthesis. Therefore, the method for producing exosomes according to the present invention is suitable for the production of exosomes having enhanced bioactivity, such as increased elastin synthesis, and the exosomes produced according to the method can be effectively used as an active ingredient in a cosmetic composition for skin elasticity improvement, wrinkle reduction and/or anti-aging.

### Example 6: Evaluation 2 of Effect of Pre-Treated Exosomes on Increase in Elastic Synthesis

The elastin synthesis increasing effects of the exosomes produced using SFM medium (hereinafter referred to as “SFM exosomes”) and the exosomes produced using EDM medium (hereinafter referred to as “EDM exosomes”) in a culture and conditioned medium collection process for exosome production (see FIG. 7) after pre-treatment with TNF-α and interferon-γ (IFN-γ) were evaluated as follows. Both of the SFM exosomes and the EDM exosomes were isolated according to Example 2-2. To this end, experimental groups were established as follows:

(1) Negative control group (control): an experimental group in which human dermal fibroblasts were not treated with exosomes;

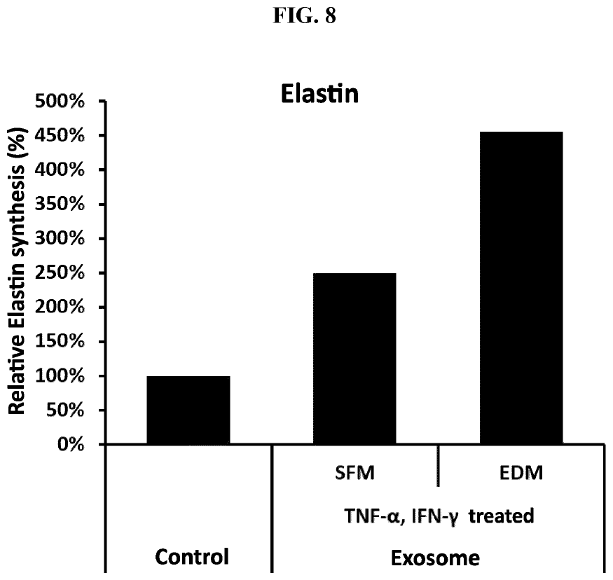

(2) SFM exosome-treated group: an experimental group in which human dermal fibroblasts were treated with the SFM exosomes at a concentration of 1.09×10¹¹ particles/mL (indicated with “SFM” in FIG. 8); and

(3) EDM exosome-treated group: an experimental group in which human dermal fibroblasts were treated with the EDM exosomes at a concentration of 1.09−10¹¹ particles/mL (indicated with “EDM” in FIG. 8).

Human dermal fibroblasts were suspended in EDF growth medium (purchased from CEFO Co., Ltd., Seoul, Korea) containing 10% growth supplement and 1% penicillin-streptomycin, and then seeded into a 48-well plate at a density of 3.0×10⁴ cells/well and cultured in an incubator at 37° C. under 5% CO₂ for 24 hours.

Thereafter, the human dermal fibroblasts were treated according to the condition of each of experimental groups (1) to (3) above, and the human dermal fibroblasts were cultured in an incubator at 37° C. under 5% CO₂ for 72 hours. After 72 hours, the culture supernatant of the human dermal fibroblasts was collected, and the amount of elastin synthesis in the culture supernatant was measured using an elastin ELISA kit (purchased from CUSABIO). The measured amount of elastin synthesis was normalized by the final number of human dermal fibroblasts measured with an MTT assay kit (purchased from Sigma).

As shown in FIG. 8, the amount of elastin synthesis in experimental group (3), in which human dermal fibroblasts were treated with the EDM exosomes, remarkably increased compared to that in experimental group (2) in which human dermal fibroblasts were treated with the SFM exosomes.

From these results, it can be seen that the EDM exosomes produced using EDM medium in the culture and conditioned medium collection process for exosome production after pre-treatment with TNF-α and interferon-γ (IFN-γ) have an excellent effect of increasing elastin synthesis. Therefore, the EDM exosomes are superior to the SFM exosomes in view that they have much enhanced bioactivity related to increased elastin synthesis, for example, skin elasticity improvement, wrinkle reduction and/or anti-aging effects. These EDM exosomes having enhanced skin elasticity improvement, wrinkle reduction and/or anti-aging effects can be economically and efficiently produced in high yield, and thus are useful commercially and/or clinically.

Therefore, the EDM exosomes having an enhanced effect of increasing elastin synthesis according to the present invention can be effectively used as an active ingredient in a cosmetic composition for skin elasticity improvement, wrinkle reduction and/or antiaging.

Although the present invention has been described with reference to the embodiments, the scope of the present invention is not limited to these embodiments. Any person skilled in the art will appreciate that various modifications and changes are possible without departing from the spirit and scope of the present invention and these modifications and changes also fall within the scope of the present invention.

We claim:

1. A method for increasing exosome production, the method comprising culturing animal cells in a medium containing TNF-α and interferon-γ.

2. The method of claim 1, wherein each of TNF-α and interferon-γ is contained in the medium at a concentration of 1 ng/mL to 100 ng/mL.

3. The method of claim 2, wherein the culturing is performed for 12 hours to 48 hours.

4. The method of claim 1, further comprising:
washing the animal cells after the culturing;
culturing the animal cells in a serum-free medium;
collecting a conditioned medium of the animal cells; and
isolating exosomes from the collected conditioned medium.

5. The method of claim 4, wherein the serum-free medium is supplemented with a platelet lysate.

6. A method for enhancing bioactivity of exosomes, the method comprising culturing animal cells in a medium containing TNF-α and interferon-γ.

7. The method of claim 6, wherein each of TNF-α and interferon-γ is contained in the medium at a concentration of 1 ng/mL to 100 ng/mL.

8. The method of claim 7, wherein the culturing is performed for 12 hours to 48 hours.

9. The method of claim 6, wherein the enhancement of bioactivity of exosomes is enhancement in increasing elastin synthesis of exosomes.

10. The method of claim 9, wherein the enhancement of bioactivity of exosomes is enhancement of at least one of a skin elasticity improvement effect and a wrinkle reduction effect.

11. A medium for increasing production of exosomes derived from animal cells, the medium containing TNF-α and interferon-γ.

12. The medium of claim 11, wherein each of TNF-α and interferon-γ is contained in the medium at a concentration of 1 ng/mL to 100 ng/mL.

13. A medium for enhancing bioactivity of exosomes derived from animal cells, the medium containing TNF-α and interferon-γ.

14. The medium of claim 13, wherein each of TNF-α and interferon-γ is contained in the medium at a concentration of 1 ng/mL to 100 ng/mL.

15. The medium of claim 13, wherein the enhancement of bioactivity of exosomes is enhancement in increasing elastin synthesis of exosomes.

16. The medium of claim 15, wherein the enhancement of bioactivity of exosomes is enhancement of at least one of a skin elasticity improvement effect and a wrinkle reduction effect.

17. A method for increasing elastin synthesis in a subject, the method comprising steps of:
obtaining exosomes having an enhanced effect of increasing elastin synthesis from a conditioned medium of animal cells by culturing the animal cells in a medium containing TNF-α and interferon-γ;
preparing a composition comprising the exosomes as an active ingredient; and
treating the subject with the composition.

18. The method of claim 17, wherein the composition is administered to the subject.

19. The method of claim 17, wherein the composition is applied to a skin of the subject.

20. The method of claim 17, wherein the exosomes are obtained by further performing steps comprising:
washing the animal cells after the culturing;
culturing the animal cells in a serum-free medium;
collecting a conditioned medium of the animal cells; and
isolating exosomes from the collected conditioned medium.

21. The method of claim 20, wherein the serum-free medium is supplemented with a platelet lysate.

22. The method of claim 17, wherein the composition enhances at least one of a skin elasticity improvement effect and a wrinkle reduction effect.
